(19) United States
(12) Patent Application Publication
(10) Pub. No.: US 2019/0032053 A1
(43) Pub. Date: Jan. 31, 2019

(54) SYNTHETIC GUIDE RNA FOR CRISPR/CAS ACTIVATOR SYSTEMS

(71) Applicant: SIGMA-ALDRICH CO. LLC, ST. LOUIS, MO (US)

(72) Inventors: Qingzhou Ji, St. Louis, MO (US); Brian Ward, St. Louis, MO (US); Andrew Ravanelli, St. Louis, MO (US)

(21) Appl. No.: 16/044,177

(22) Filed: Jul. 24, 2018

Related U.S. Application Data
(60) Provisional application No. 62/539,314, filed on Jul. 31, 2017.

(51) Int. Cl. C12N 15/113; C12N 15/861

(52) U.S. Cl. CPC C12N 15/113; C12N 15/861; C12N 2310/20; C12N 2310/315; C12N 2310/3521; C12N 2320/51; C12N 2310/321; C12N 2310/322; C12N 2310/346

(57) ABSTRACT

Compositions comprising synthetic two-part aptamer-containing guide RNAs and methods of using said synthetic two-part aptamer-containing guide RNAs with CRISPR/Cas activator systems.

### CROSS-REFERENCE TO RELATED APPLICATIONS

This application claims priority to U.S. Provisional Application Ser. No. 62/539,314, filed Jul. 31, 2017, the disclosure of which is hereby incorporated by reference in its entirety.

### SEQUENCE LISTING

This application contains a Sequence Listing that has been submitted in ASCII format via EFS-Web and is hereby incorporated by reference in its entirety. The ASCII copy, created on Jul. 24, 2018, is named 602192_SequenceListing_ST25.txt, and is 13 kilobytes in size.

### FIELD

The present disclosure relates to synthetic two-part guide RNAs comprising RNA aptamer sequences and uses thereof.

### BACKGROUND

The CRISPR/Cas9 synergistic activation mediator (SAM) system (Konermann et al., Nature, 2015, 517(7536):583-588) provides a platform for high-level transcriptional activation by combining the VP64-dCas9 artificial transcription factor with an aptamer-sgRNA that recruits additional transcriptional co-activators. Because of the additional aptamer sequences, chemical synthesis of single SAM-gRNAs remains challenging. Thus, the use of sgRNA may limit the ease of use and efficiency of CRISPR/Cas9 SAM systems. What is needed, therefore, is a two-part aptamer-containing gRNA system, which can be readily and efficiently produced, for use with CRISPR/Cas activator systems.

### SUMMARY

Among the various aspects of the disclosure is the provision of synthetic two-part guide RNAs (gRNAs), wherein each two-part gRNA comprises (a) a clustered regularly interspersed short palindromic repeats (CRISPR) RNA (crRNA) and (b) a transacting crRNA (tracrRNA). Each crRNA comprises a 5′ sequence that is complementary to a target sequence in chromosomal DNA and a 3′ sequence that is capable of base pairing with a portion of the tracrRNA, and each tracrRNA comprises a 5′ tetraloop and at least one stem-loop, and the 5′ tetraloop and/or at least one stem-loop is modified to contain at least one hairpin-forming RNA aptamer sequence.

In general, the at least one hairpin-forming RNA aptamer sequence can be MS2 sequence, PP7 sequence, com sequence, box B sequence, histone mRNA 3′ sequence, AU-rich element (ARE) sequence, or variants thereof, and the at least one hairpin-forming RNA aptamer sequence can be located in the 5′ tetraloop, in the at least one stem-loop, and/or at the 3′ end of the tracrRNA.

In some embodiments, the at least one stem-loop of the tracrRNA comprises stem-loop 1, stem-loop 2, and stem-loop 3, and the at least one hairpin-forming RNA aptamer sequence can be located in the 5′ tetraloop and/or in stem-loop 2. In some instances, the 5′ tetraloop and/or stem-loop 2 can further comprises an extension sequence, which can range from about 2 nucleotides to about 30 nucleotides. In certain embodiments, the crRNA further comprises a sequence that is capable of base paring with the extension sequence in the 5′ tetraloop or a portion of the extension sequence in the 5′ tetraloop of the tracrRNA.

In certain embodiments, the crRNA is chemically synthesized and the tracrRNA is enzymatically synthesized in vitro.

Also provided herein are nucleic acids encoding the tracrRNAs as described above.

Another aspect of the present disclosure encompasses methods for targeted transcription activation, targeted transcription repression, targeted epigenome modification, targeted genome modification, or targeted genomic locus visualization in a eukaryotic cell. The methods comprise introducing into the eukaryotic cell (a) a synthetic two-part gRNA as defined above, (b) at least one RNA aptamer binding protein associated with at least one functional domain or nucleic acid encoding the at least one RNA aptamer binding protein associated with at least one functional domain, and (c) at least one CRISPR/Cas protein or nucleic acid encoding the at least one CRISPR/Cas protein, wherein interactions between (a), (b), (c), and the target sequence in chromosomal DNA leads to targeted transcription activation, targeted transcription repression, targeted epigenome modification, targeted genome modification, or targeted genomic locus visualization in the eukaryotic cell. The method can further comprise introducing one or more additional crRNAs, wherein each additional crRNA comprises a different 5′ sequence but a universal 3′ sequence.

In some embodiments, the at least one RNA aptamer binding protein can be MCP, PCP, Com, N22, SLBP, or FXR1, and the at least one functional domain associated with the at least one RNA aptamer binding protein can be a transcription activation domain, a transcription repressor domain, an epigenetic modification domain, a marker domain, or combination thereof. In certain iterations, the transcription activation domain can be VP16 activation domain, VP64 activation domain, VP160 activation domain, p65 activation domain from NFκB, or heat-shock factor 1 (HSF1) activation domain; the transcription repressor domain can be Kruppel-associated box (KRAB) repressor domain; the epigenetic modification domain can be p300 histone acetyltransferase, activation-induced cytidine deaminase (AID), APOBEC cytidine deaminase, TET methylcytosine dioxygenase, or has nucleosome interacting activity; and the marker domain can be a fluorescent protein, a purification, or an epitope tag.

In various embodiments, the at least one CRISPR/Cas protein can be a CRISPR/Cas nuclease or a catalytically inactive CRISPR/Cas protein linked to a non-CRISPR/Cas nuclease domain. In some instances, the method can further comprise introducing into the eukaryotic cell a donor polynucleotide comprising at least one donor sequence.

In other embodiments, the at least one CRISPR/Cas protein can be a catalytically inactive CRISPR/Cas protein linked to a non-nuclease domain, wherein the non-nuclease domain can be a transcription activation domain, a transcription repressor domain, or an epigenetic modification domain.

In particular embodiments, the at least one CRISPR/Cas protein can be a type II Cas9 protein.

In various embodiments, the eukaryotic cell can be in vitro or in vivo. In other situations, the eukaryotic cell can be a mammalian cell, such as a human cell.

Other features and aspects of the disclosure are described in detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents the sequence and secondary structure of a two-part crRNA (SEQ ID NO:38) and aptamer-tracrRNA (SEQ ID NO:39) (design #1). The tetraloop extension in the tracrRNA is underlined and the MS2 stem-lop structures in the tracrRNA are bolded.

FIG. 2A shows targeted activation of the POU5F1 gene with the CRISPR two-part synthetic crRNA and aptamer-tracrRNA system in HEK293 cells.

FIG. 2B presents targeted activation of the IL1B gene with the CRISPR two-part synthetic crRNA and aptamer-tracrRNA system in HEK293 cells.

### DETAILED DESCRIPTION

The present disclosure provides synthetic two-part guide RNAs comprising aptamer sequences for use with CRISPR/Cas activator systems. The two-part system comprises a target-specific crRNA and a universal aptamer-tracrRNA. The short, target-specific crRNA can be readily chemically synthesized, and the longer universal aptamer-tracrRNA can be enzymatically synthesized in vitro and stored for later use. Alternatively, both the crRNA and the tracrRNA can be chemically synthesized. Also provided herein are compositions comprising the synthetic two-part guide RNAs, kits comprising the synthetic two-part guide RNAs, and methods for using the synthetic two-part guide RNAs.

### (I) Synthetic Two-Part Guide RNAs

One aspect of the present disclosure provides synthetic two-part guide RNAs (gRNAs) comprising or consisting of a CRISPR RNA (crRNA) and a transacting crRNA (tracrRNA), wherein the tracrRNA comprises at least one hairpin-forming RNA aptamer sequence.

(a) crRNA

The synthetic two-part gRNA disclosed herein comprise a crRNA. Each crRNA comprises a 5′ sequence (i.e., spacer sequence) that is complementary to a target sequence in chromosomal DNA and a 3′ sequence that is capable of base pairing with a portion of the tracrRNA. The 5′ spacer sequence is different in each crRNA, whereas the 3′ sequence generally can be the same in each crRNA.

The spacer sequence at the 5′ end of the crRNA is complementary to a target sequence (i.e., protospacer sequence) in chromosomal DNA such that the crRNA can hybridize with the target sequence. The target sequence has no sequence limitation except that the sequence is adjacent to a protospacer adjacent motif (PAM). For example, PAM sequences for various CRISPR/Cas proteins include 5′-NGG (SpCas9), 5′-NGGNG (St3Cas9), 5′-NNAGAAW (St1Cas9), 5′-NNGRRT (SaCas9), 5′NNNNGATT (NmCas9), and 5′-TTTN (AsCpf1), wherein N is defined as any nucleotide, and W is defined as either A or T.

The length of the 5′ spacer sequence having complementarity to the target sequence can range from about 10 nucleotides to more than about 25 nucleotides. In some embodiments, the region of base pairing between the spacer sequence of the crRNA and the target sequence can be 14, 15, 16, 17, 18, 19, 20, 21, 22, or 23 nucleotides in length. In specific embodiments, the region of base pairing between the spacer sequence of the crRNA and the target sequence can be 19, 20, or 21 nucleotides in length. For example, the spacer sequence of a SpCas9 crRNA can comprise N₂₀ or GN₁₇₋₂₀GG. In general, the sequence identity between the spacer sequence of the crRNA and the target sequence can be at least about 80%, at least about 85%, at least about 90%, at least about 95%, or at least about 99%. Those skilled in the art appreciate that increased sequence identity with the target sequence can result in fewer off target effects.

The crRNA also comprises a 3′ sequence that is capable of base pairing with sequence near the 5′ end of the tracrRNA. The length of the 3′ sequence of the crRNA can range from about 5 nucleotides to about 25 nucleotides. In some embodiments, the length of the 3′ sequence in the crRNA can range from about 9 nucleotides to about 15 nucleotides. In specific embodiments, the length of the 3′ sequence in the crRNA can be about 12 nucleotides. The sequence identity between the 3′ sequence in the crRNA and the complementary tracrRNA sequence generally is at least about 50%. Thus, the base pairing between the crRNA and tracrRNA can comprise stretches of at least two contiguous base pairs (e.g., two or more stretches of three or more contiguous base pairs separated by unhybridized sequence).

In some embodiments, the crRNA can further comprise additional 3′ sequence that is capable of base paring with an extension sequence in the 5′ tetraloop or a portion of the extension sequence in the 5′ tetraloop of the tracrRNA (see below). The additional sequence in the crRNA can range from about 2 nucleotides to about 30 nucleotides. The sequence identity between the additional sequence in the crRNA and the extension sequence in the 5′ tetraloop is generally at least about 50%.

In general, the crRNA is chemically synthesized using solid-phase synthesis technologies. As such, the crRNA can comprise standard ribonucleotides or modified ribonucleotides. Modified ribonucleotides include base modifications (e.g., pseudouridine, 2-thiouridine, N6-methyladenosine, and the like) and/or sugar modifications (e.g., 2′-O-methy, 2′-fluoro, 2′-amino, locked nucleic acid (LNA), and so forth). The backbone of the crRNA can also be modified to comprise phosphorothioate or boranophosphate linkages or peptide nucleic acids. The 5′ and 3′ ends of the crRNA can be conjugated to functional moieties such as fluorescent dyes (e.g., FAM, TMR, Cy3, Cy5, Texas Red, Oregon Green, Alexa Fluors, Halo tags, and the like), detection tags (e.g., biotin, digoxigenin, quantum dots, gold particles, etc.), polymers, proteins, and the like. Those skilled in the art will appreciate that the crRNA can also be synthesized enzymatically in vitro.

(b) Aptamer-tracrRNA

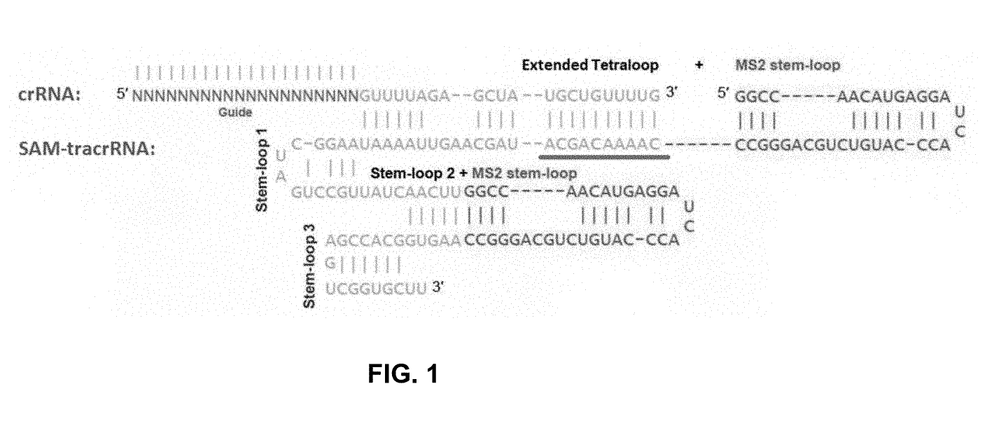

The synthetic two-part gRNA disclosed herein also comprises a tracrRNA that comprises at least one hairpin-forming RNA aptamer sequence. The tracrRNAs disclosed herein comprise, from 5′ to 3′, a 5′ tetraloop, a sequence capable of base pairing with the crRNA, at least one internal stem-loop, and a single-stranded 3′ sequence. The at least one internal stem-loop can comprise one stem-loop, two stem-loops, three stem-loops, four stem-loops, 5 stem-loops, or more than five stem-loops. In specific embodiments, the at least one internal stem-loop can comprise stem-loop 1, stem-loop 2, and stem-loop 3 (see FIG. 1). The internal stem-loop(s) of the tracrRNA can form a secondary structure that interacts with the CRISPR/Cas protein to form a stable ternary DNA-gRNA-protein complex. The sequence and/or secondary structure of the tracrRNA can and will vary depending, for example, on the identity of CRISPR/Cas protein with which it is designed to complex (e.g., SpCas9, SaCas9, CjCas9, and the like).

The tracrRNAs disclosed herein further comprise at least one hairpin-forming RNA aptamer sequence. The at least one hairpin-forming RNA aptamer sequence can be located in the 5′ tetraloop, the at least one internal stem-loop, and/or the 3′ end of the tracrRNA. In some embodiments, the at least one hairpin-forming RNA aptamer sequence can be located in the 5′ tetraloop. In other embodiments, at least one hairpin-forming RNA aptamer sequence can be located in at least one of the internal stem-loops of the tracrRNA. For example, the at least one hairpin-forming RNA aptamer sequence can be located in stem-loop 2. In further embodiments, at least one hairpin-forming RNA aptamer sequence can be located in the 3′ end of the tracrRNA. In still other embodiments, hairpin-forming RNA aptamer sequences can be located in the 5′ tetraloop and in stem-loop 2. In alternate embodiments, hairpin-forming RNA aptamer sequences can be located in the 5′ tetraloop and in the 3′ end of the tracrRNA. In further embodiments, hairpin-forming RNA aptamer sequences can be located in the 5′ tetraloop, in stem-loop 2, and in the 3′ end of the tracrRNA.

A variety of one hairpin-forming RNA aptamer sequences can be included in the tracrRNAs disclosed herein. The hairpin-forming RNA aptamer sequence can comprise multiples of or combinations of any of the aptamer sequences listed below. In some embodiments, the at least one hairpin-forming RNA aptamer sequence can be MS2 aptamer sequence or variant thereof that binds MS2 bacteriophage coat protein (MCP) (Lowary et al., Nuc Acid Res, 1987, 15(24):10483-10493). MS2 variants include F5 and F6 aptamers (Parrott et al., Nucl Acids Res, 2000, 28(2):489-497). In other embodiments, the at least one hairpin-forming RNA aptamer sequence can be PP7 sequence that binds PP7 bacteriophage coat protein (PCP) (Lim et al., J Biol Chem, 2001, 276(25):22507-22513). In alternate embodiments, the at least one hairpin-forming RNA aptamer sequence can be com sequence that binds Mu bacteriophage Com protein (Hattman, Pharmacol & Ther, 1999, 84(3):367-388). In further embodiments, the at least one hairpin-forming RNA aptamer sequence can be box B sequence that binds lambda bacteriophage N22 protein (Daigle et al., Nat Methods, 2007, 4:633-636). In further embodiments, the at least one hairpin-forming RNA aptamer sequence can be AU-rich element (ARE) sequence that binds Fragile X mental retardation syndrome-related protein 1 (FXR1) (Vasudevan et al., Science, 2007, 318(5858):1931-1934). In still other embodiments, the at least one hairpin-forming RNA aptamer sequence can be histone mRNA 3′ sequence that binds stem-loop binding protein (SLBP). In yet other embodiments, the at least one hairpin-forming RNA aptamer sequence can be a sequence that binds a protein from a bacteriophage chosen from AP205, BZ13, f1, f2, fd, fr, ID2, JP34/GA, JP501, JP34, JP500, KU1, M11, M12, MX1, NL95, PP7, ϕCb5, ϕCb8r, ϕCb12r, ϕCb23r, Qβ, R17, SP-β, TW18, TW19, or VK.

The length of the hairpin-forming RNA aptamer sequence introduced into the at least one loop of the tracrRNA can and will vary depending upon the identity of the hairpin-forming RNA aptamer sequence. For example, a MS2 aptamer sequence can be about 34 nucleotides in length. In various embodiments, the hairpin-forming RNA aptamer sequence can range in length from about 10 nucleotides to about the 50 nucleotides.

In embodiments in which the at least one hairpin-forming RNA aptamer sequence is located in the 5′ tetraloop and/or one or more of the internal stem-loops, the 5′ tetraloop and/or the internal stem-loop(s) can further comprise an extension sequence. In some embodiments, the at least one hairpin-forming RNA aptamer sequence is located in the 5′ tetraloop, and the 5′ tetraloop further comprises the extension sequence. In such embodiments, the crRNA can further comprise a sequence that is complementary to the extension sequence in the 5′ tetraloop or a portion of the extension sequence in the 5′ tetraloop (a non-limiting example is diagrammed in FIG. 1).

The extension sequence can range in length from about 2 nucleotides to about 30 nucleotides. In some embodiments, the extension sequence can range in length from about 3 nucleotides to about 25, or from about 5 nucleotides to about 25 nucleotides. In various embodiments, the extension sequence can comprise about 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In specific embodiments, the extension sequence can comprise 4 nucleotides, 6 nucleotides, 8 nucleotides, 10 nucleotides, 12 nucleotides, 14 nucleotides, 16 nucleotides, 18 nucleotides, or 20 nucleotides.

The total length of the aptamer-tracrRNA can and will vary depending upon the identity of the RNA aptamer sequence, the number of RNA aptamers sequences present in the tracrRNA, as well as the length of the optional extension sequence(s). In general, the aptamer-tracrRNA can range in length from about 80 nucleotides to about 300 nucleotides. In various embodiments, the total length of the aptamer-tracrRNA can range up to about 120 nucleotides, up to about 125 nucleotides up to about 150 nucleotides, up to about 175 nucleotides, up to about 200 nucleotides, up to about 225 nucleotides, up to about 250 nucleotides, up to about 275 nucleotides, or up to about 300 nucleotides.

In some embodiments, the tracrRNA can be enzymatically synthesized in vitro. For example, DNA encoding the tracrRNA can be operably linked to a promoter sequence that is recognized by a phage RNA polymerase, as detailed below in section (IV). As such, the tracrRNA comprises standard ribonucleotides (or those that can be incorporated by the enzyme used in vitro). In other embodiments, the tracrRNA can be chemically synthesized and can comprise standard ribonucleotides, modified ribonucleotides, standard phosphodiester linkages, or modified linkages (e.g., (phosphorothioate, boranophosphate, or peptide nucleic acid linkages).

### (II) Compositions

Another aspect of the present disclosure encompasses compositions comprising or consisting of 1) a synthetic two-part guide RNA as described above in section (I) and at least one RNA aptamer binding protein, or 2) a synthetic two-part guide RNA, at least one RNA aptamer binding protein, and at least one CRISPR/Cas protein. In some embodiments, the composition can comprise nucleic acids encoding the at least one RNA aptamer binding protein and/or the CRISPR/Cas protein (see section (IV) below).

(a) RNA Aptamer Binding Proteins

The compositions comprise at least one RNA aptamer binding protein. RNA aptamer binding proteins bind the one or more aptamer sequences located in the tracrRNA of the synthetic two-part guide RNA. The RNA aptamer protein generally is associated with at least one functional domain. The at least one functional domain can be a transcription activation domain, a transcription repressor domain, an epigenetic modification domain, a marker domain, or combination thereof.

(i) RNA Aptamer Binding Proteins

Non-limiting examples of suitable RNA aptamer binding proteins include MS2 coat protein (MCP), PP7 bacteriophage coat protein (PCP), Mu bacteriophage Com protein, lambda bacteriophage N22 protein, stem-loop binding protein (SLBP), and Fragile X mental retardation syndrome-related protein 1 (FXR1). In other embodiments, the RNA aptamer binding protein can be a protein from a bacteriophage chosen from AP205, BZ13, f1, f2, fd, fr, ID2, JP34/GA, JP501, JP34, JP500, KU1, M11, M12, MX1, NL95, PP7, ϕCb5, ϕCb8r, ϕCb12r, ϕCb23r, Qβ, R17, SP-β, TW18, TW19, or VK.

(ii) Functional Domains

The RNA aptamer binding protein is associated with at least one functional domain, wherein the functional domain is a transcription activation domain, a transcription repressor domain, an epigenetic modification domain, a marker domain, or combination thereof.

In some embodiments, the at least one functional domain can be a transcription activation domain. Suitable transcription activation domains include, without limit, herpes simplex virus VP16 domain, VP64 (which is a tetrameric derivative of VP16), VP160 10×VP16), p65 activation domain from NFκB, heat-shock factor 1 (HSF1) activation domain, MyoD1 activation domain, GCN4 peptide, 10×GCN4, viral R transactivator (Rta), VPR (a fusion of VP64-p65-Rta), p53 activation domains 1 and 2, CREB (cAMP response element binding protein) activation domains, E2A activation domains, or nuclear factor of activated T-cells (NFAT) activation domains.

In other embodiments, the at least one functional domain can be a transcription repressor domain. Non-limiting examples of suitable transcription repressor domains include Kruppel-associated box (KRAB) repressor domains, inducible cAMP early repressor (ICER) domains, YY1 glycine rich repressor domains, Sp1-like repressors, E(spI) repressors, IκB repressor, or methyl-CpG binding protein 2 (MeCP2) repressor domain.

In further embodiments, the at least one functional domain can be an epigenetic modification domain. Epigenetic modification domains can alter DNA or chromatin structure (and may or may not alter DNA sequence). Non-limiting examples of suitable epigenetic modification domains include those with DNA methyltransferase activity (e.g., cytosine methyltransferase), DNA demethylase activity, DNA deamination (e.g., cytosine deaminase, adenosine deaminase, guanine deaminase), DNA amination, DNA oxidation activity, DNA helicase activity, histone acetyltransferase (HAT) activity (e.g., HAT domain derived from E1A binding protein p300), histone deacetylase activity, histone methyltransferase activity, histone demethylase activity, histone kinase activity, histone phosphatase activity, histone ubiquitin ligase activity, histone deubiquitinating activity, histone adenylation activity, histone deadenylation activity, histone SUMOylating activity, histone deSUMOylating activity, histone ribosylation activity, histone deribosylation activity, histone myristoylation activity, histone demyristoylation activity, histone citrullination activity, histone alkylation activity, histone dealkylation activity, histone oxidation activity, or nucleosome interacting/remodeling activity. In specific embodiments, the epigenetic modification domain can comprise cytidine deaminase activity, histone acetyltransferase activity, or DNA methyltransferase activity. In particular embodiments, the epigenetic modification domain can be p300 histone acetyltransferase, activation-induced cytidine deaminase (AID), APOBEC cytidine deaminase, or TET methylcytosine dioxygenase.

In still other embodiments, the at least one functional domain can be a marker domain. Marker domains include fluorescent proteins and purification or epitope tags. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato). Non-limiting examples of suitable purification or epitope tags include 6× His, FLAG®, HA, GST, Myc, and the like.

(iii) Association Between RNA Binding Protein and Functional Domain(s)

The RNA aptamer binding protein is associated with at least one functional domain. In some embodiments, the RNA aptamer binding protein can be associated with one functional domain. In other embodiments, the RNA aptamer binding protein can be associated with two functional domains. In further embodiments, the RNA aptamer binding protein can be associated with three functional domains. In additional embodiments, the RNA aptamer binding protein can be associated with four two functional domains or more than four functional domains. The functional domains associated with on RNA aptamer binding protein can have the same function or they can have different functions. For example, the RNA aptamer binding protein can be associated with two transcription activation domains, two epigenetic modification domains, a transcription activation domain and an epigenetic modification domain, at least one transcription activation domain and a marker domain, and so forth.

The RNA aptamer binding protein can be associated with the at least one functional domains directly via chemical bonds or indirectly via linkers. The chemical bond can be covalent (e.g., peptide bond, ester bond, and the like). Alternatively, the chemical bond can be non-covalent (e.g., ionic, electrostatic, hydrogen, hydrophobic, Van der Waals interactions, or π-effects). In some embodiments, the RNA aptamer binding protein can be associated with the at least one functional domains via noncovalent protein-protein, protein-RNA, or protein-DNA interactions. In certain embodiments, the RNA aptamer binding protein and the associated domain can be linked directly via peptide bond, thereby forming a fusion protein.

In other embodiments, the RNA aptamer binding protein can be associated with the at least one functional domains via linkers. A linker is a chemical group that connects one or more other chemical groups via at least one covalent bond. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme.

In further embodiments, the RNA aptamer binding protein can be linked to the at least one functional domain via peptide linkers. The peptide linker can be a flexible amino acid linker (e.g., comprising small, non-polar or polar amino acids). Non-limiting examples of flexible linkers include LEGGGS (SEQ ID NO:1), TGSG (SEQ ID NO:2), GGSGGGSG (SEQ ID NO:3), and (GGGGS)₁₋₄ (SEQ ID NO:4. Alternatively, the peptide linker can be a rigid amino acid linker. Such linkers include (EAAAK)₁₋₄ (SEQ ID NO:5), A(EAAAK)₂₋₅A (SEQ ID NO:6), and PAPAP (SEQ ID NO:7). Examples of suitable linkers are well known in the art and programs to design linkers are readily available (Crasto et al., Protein Eng., 2000, 13(5):309-312). In certain embodiments, the RNA aptamer binding protein and the associated domain can be linked directly via a peptide linker, thereby forming a fusion protein.

The at least one functional domain can be associated with the N-terminus, the C-terminus, and/or an internal location of the RNA aptamer binding protein.

(iv) Optional Nuclear Localization Signal and/or Cell Penetrating Peptide

The RNA aptamer binding protein can further comprise at least one nuclear localization signal (NLS) and/or cell penetrating peptide (CPP). Non-limiting examples of nuclear localization signals include PKKKRKV (SEQ ID NO:8), PKKKRRV (SEQ ID NO:9), KRPAATKKAGQAKKKK (SEQ ID NO:10), YGRKKRRQRRR (SEQ ID NO:11), RKKRRQRRR (SEQ ID NO:12), PAAKRVKLD (SEQ ID NO:13), RQRRNELKRSP (SEQ ID NO:14), VSRKRPRP (SEQ ID NO:15), PPKKARED (SEQ ID NO:16), PQPKKKPL (SEQ ID NO:17), SALIKKKKKMAP (SEQ ID NO:18), PKQKKRK (SEQ ID NO:19), RKLKKKIKKL (SEQ ID NO:20), REKKKFLKRR (SEQ ID NO:21), KRKGDEVDGVDEVAKKKSKK (SEQ ID NO:22), RKCLQAGMNLEARKTKK (SEQ ID NO:23), NQSSNFGPMKGGNFGGRSSGPYGGGGQYFAKPRNQGGY (SEQ ID NO:24), and RMRIZFKNKGKDTAELRRRRVEVSVELRKAKKDEQILKRRNV (SEQ ID NO:25). Examples of suitable cell penetrating peptides include, without limit, GRKKRRQRRRPPQPKKKRKV (SEQ ID NO:26), PLSSIFSRIGDPPKKKRKV (SEQ ID NO:27), GALFLGWLGAAGSTMGAPKKKRKV (SEQ ID NO:28), GALFLGFLGAAGSTMGAWSQPKKKRKV (SEQ ID NO:29), KETWWETWWTEWSQPKKKRKV (SEQ ID NO:30), YARAAARQARA (SEQ ID NO:31), THRLPRRRRRR (SEQ ID NO:32), GGRRARRRRRR (SEQ ID NO:33), RRQRRTSKLMKR (SEQ ID NO:34), GWTLNSAGYLLGKINLKALAALAKKIL (SEQ ID NO:35), KALAWEAKLAKALAKALAKHLAKALAKALKCEA (SEQ ID NO:36), and RQIKIWFQNRRMKWKK (SEQ ID NO:37).

The at least one nuclear localization signal and/or cell penetrating peptide can be associated with the N-terminus, the C-terminus, and/or an internal location of the RNA aptamer binding protein and/or the at least one functional domain.

(b) CRISPR/Cas Proteins

The composition can further comprise a CRISPR/Cas protein. In some embodiments, the CRISPR/Cas protein has nuclease activity and is capable of cleaving both strands of a double-stranded DNA sequence (i.e., generates a double-stranded break). In other embodiments, the CRISPR/Cas protein has non-nuclease activity (i.e., is a catalytically inactive CRISPR/Cas protein linked to a non-nuclease domain). Suitable non-nuclease domains include transcription activation domains, transcription repressor domains, and epigenetic modification domains.

In general, the CRISPR/Cas protein and the RNA aptamer binding protein are chosen to work in concert. For example, a CRISPR/Cas protein having nuclease activity could be used with an RNA aptamer binding protein associated with a domain having nucleosome interacting activity. Similarly, a catalytically inactive CRISPR/Cas protein linked to a transcription activation domain could be used with an RNA aptamer binding protein associated a transcription activation domain. Those skilled in the art can appreciate the numerous possibilities.

(i) CRISPR/Cas Proteins with Nuclease Activity

CRISPR/Cas Nucleases. The CRISPR/Cas protein having nuclease activity can be derived from a type I (i.e., IA, IB, IC, ID, IE, or IF), type II (i.e., IIA, IIB, or IIC), type III (i.e., IIIA or IIIB), or type V CRISPR system, which are present in various bacteria and archaea. For example, the CRISPR/Cas system can be from Streptococcus sp. (e.g., S. pyogenes, S. thermophilus, S. pasteurianus), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lachnospiraceae sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., Thermosipho sp., or Verrucomicrobia sp. In other embodiments, the CRISPR/Cas nuclease can be derived from an archaeal CRISPR system, a CRISPR/CasX system, or a CRISPR/CasY system (Burstein et al., Nature, 2017, 542(7640):237-241).

In some embodiments, the CRISPR/Cas nuclease can be derived from a type I CRISPR/Cas system. In other embodiments, the CRISPR/Cas nuclease can be derived from a type II CRISPR/Cas system. In still other embodiments, the CRISPR/Cas nuclease can be derived from a type III CRISPR/Cas system. In further particular embodiments, the CRISPR/Cas nuclease can be derived from a type V CRISPR/Cas system.

The CRISPR/Cas nuclease can be a wild type or naturally-occurring protein. Alternatively, the CRISPR/Cas protein can be engineered to have improved specificity, altered PAM specificity, decreased off-target effects, increased stability, and the like.

Non-limiting examples of suitable CRISPR/Cas nucleases include Cas proteins (e.g., Cas9, Cas1, Cas2, Cas3, and the like), Cpf proteins, C2c proteins (e.g., C2c1, C2c2, Cdc3), Cmr proteins, Csa proteins, Csb proteins, Csc proteins, Cse proteins, Csf proteins, Csm proteins, Csn proteins, Csx proteins, Csy proteins, Csz proteins, and derivatives or variants thereof. In specific embodiments, the CRISPR/Cas nuclease can be a type II Cas9 protein, a type V Cpf1 protein, or derivative thereof.

In some embodiments, the CRISPR/Cas nuclease can be Streptococcus pyogenes Cas9 (SpCas9), Streptococcus thermophilus Cas9 (St1Cas9 or St3Cas9), or Streptococcus pasteurianus (SpaCas9). In other embodiments, the CRISPR/Cas nuclease can be Campylobacter jejuni Cas9 (CjCas9). In alternate embodiments, the CRISPR/Cas nuclease can be Francisella novicida Cas9 (FnCas9). In yet other embodiments, the CRISPR/Cas nuclease can be a Neisseria meningitides Cas9 (NmCas9). In still other embodiments, the CRISPR/Cas nuclease can be Neisseria cinerea Cas9 (NcCas9). In further embodiments, the CRISPR/Cas nuclease can be Francisella novicida Cpf1 (FnCpf1), Acidaminococcus sp. Cpf1 (AsCpf1), or Lachnospiraceae bacterium ND2006 Cpf1 (LbCpf1).

In general, the CRISPR/Cas nuclease comprises an RNA recognition and/or RNA binding domain, which interacts with the tracrRNA. The CRISPR/Cas nuclease also comprises at least one nuclease domain having endonuclease activity. For example, a Cas9 protein comprises a RuvC-like nuclease domain and an HNH-like nuclease domain, and a Cpf1 protein comprises a RuvC-like domain and a NUC domain. CRISPR/Cas nucleases can also comprise DNA binding domains, helicase domains, RNase domains, protein-protein interaction domains, dimerization domains, as well as other domains.

In some embodiments, the CRISPR/Cas nuclease can be a CRISPR/Cas nickase in which the CRISPR/Cas nuclease has been modified to cleave only one strand of DNA. A CRISPR/Cas nickase used in combination with a pair of offset guide RNAs (i.e., a CRISPR/Cas dual nickase) can generate a double-stranded break in a double-stranded sequence. A CRISPR/Cas nuclease can be converted to a nickase by one or more mutations and/or deletions. For example, a Cas9 nickase can comprise one or more mutations in one of the nuclease domains (e.g., the RuvC-like domain or the HNH-like domain). For example, the one or more mutations can be D10A, D8A, E762A, and/or D986A in the RuvC-like domain or the one or more mutations can be H840A, H559A, N854A, N856A, and/or N863A in the HNH-like domain such that the nickase cleaves only one strand of a double stranded DNA sequence.

Catalytically Inactive CRISPR/Cas Protein Linked to a Non-CRISPR/Cas Nuclease Domain. In additional embodiments, the CRISPR/Cas protein having nuclease activity comprise a catalytically inactive CRISPR/Cas protein linked to a non-CRISPR/Cas nuclease domain. The catalytically inactive CRISPR/Cas protein has been modified by mutation and/or deletion by to lack all nuclease activity. For example, the catalytically inactive CRISPR/Cas protein can be a catalytically inactive (dead) Cas9 (dCas9) in which the RuvC-like domain comprises a D10A, D8A, E762A, and/or D986A mutation and the HNH-like domain comprises a H840A, H559A, N854A, N865A, and/or N863A mutation. Alternatively, the catalytically inactive CRISPR/Cas protein can be a catalytically inactive (dead) Cpf1 protein comprising comparable mutations in the nuclease domain.

The catalytically inactive CRISPR/Cas protein can be linked to a nuclease domain derived from a restriction endonuclease or a homing endonuclease. In specific embodiments, the nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In specific embodiments, the nuclease domain can be a FokI nuclease domain or a derivative thereof. The type II-S nuclease domain can be modified to facilitate dimerization of two different nuclease domains. For example, the cleavage domain of FokI can be modified by mutating certain amino acid residues. In specific embodiments, the FokI nuclease domain can comprise a first FokI half-domain comprising Q486E, I499L, and/or N496D mutations, and a second FokI half-domain comprising E490K, I538K, and/or H537R mutations.

The catalytically inactive CRISPR/Cas protein can be linked to the non-CRISPR/Cas nuclease domain directly via chemical bonds or indirectly via linkers. The chemical bond can be covalent (e.g., peptide bond, ester bond, and the like). Alternatively, the chemical bond can be non-covalent (e.g., ionic, electrostatic, hydrogen, hydrophobic, Van der Waals interactions, or π-effects). Suitable linkers are described above in section (II)(a)(iii). The nuclease domain can be linked to the N-terminus, the C-terminus, and/or an internal location of the catalytically inactive CRISPR/Cas protein.

Optional Protein Domains. The CRISPR/Cas protein having nuclease activity can further comprise at least one nuclear localization signal (NLS), cell penetrating peptide (CPP), and/or marker domain. The at least one NLS, CPP, and/or marker domain can be linked directly or indirectly to e N-terminus, the C-terminus, and/or an internal location of the CRISPR/Cas protein having nuclease activity.

Non-limiting examples of nuclear localization signals include PKKKRKV (SEQ ID NO:8), PKKKRRV (SEQ ID NO:9), KRPAATKKAGQAKKKK (SEQ ID NO:10), YGRKKRRQRRR (SEQ ID NO:11), RKKRRQRRR (SEQ ID NO:12), PAAKRVKLD (SEQ ID NO:13), RQRRNELKRSP (SEQ ID NO:14), VSRKRPRP (SEQ ID NO:15), PPKKARED (SEQ ID NO:16), PQPKKKPL (SEQ ID NO:17), SALIKKKKKMAP (SEQ ID NO:18), PKQKKRK (SEQ ID NO:19), RKLKKKIKKL (SEQ ID NO:20), REKKKFLKRR (SEQ ID NO:21), KRKGDEVDGVDEVAKKKSKK (SEQ ID NO:22), RKCLQAGMNLEARKTKK (SEQ ID NO:23), NQSSNFGPMKGGNFGGRSSGPYGGGGQYFAKPRNQGGY (SEQ ID NO:24), and RMRIZFKNKGKDTAELRRRRVEVSVELRKAKKDEQILKRRNV (SEQ ID NO:25). Examples of suitable cell penetrating peptides include, without limit, GRKKRRQRRRPPQPKKKRKV (SEQ ID NO:26), PLSSIFSRIGDPPKKKRKV (SEQ ID NO:27), GALFLGWLGAAGSTMGAPKKKRKV (SEQ ID NO:28), GALFLGFLGAAGSTMGAWSQPKKKRKV (SEQ ID NO:29), KETWWETWWTEWSQPKKKRKV (SEQ ID NO:30), YARAAARQARA (SEQ ID NO:31), THRLPRRRRRR (SEQ ID NO:32), GGRRARRRRRR (SEQ ID NO:33), RRQRRTSKLMKR (SEQ ID NO:34), GWTLNSAGYLLGKINLKALAALAKKIL (SEQ ID NO:35), KALAWEAKLAKALAKALAKHLAKALAKALKCEA (SEQ ID NO:36), and RQIKIWFQNRRMKWKK (SEQ ID NO:37). The marker domain can be a fluorescent protein and/or a purification or epitope tag. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato). Non-limiting examples of suitable purification or epitope tags include 6× His, FLAG®, HA, GST, Myc, and the like.

(ii) CRISPR/Cas Protein With Non-Nuclease Activity

In alternate embodiments, the CRISPR/Cas protein can have non-nuclease activity. For example, the CRISPR/Cas protein can be a catalytically inactive CRISPR/Cas protein linked to at least one non-nuclease domain. As mentioned above, the catalytically inactive CRISPR/Cas protein has been modified by mutation and/or deletion by to lack all nuclease activity. For example, the catalytically inactive CRISPR/Cas protein can be a catalytically inactive (dead) Cas9 (dCas9) in which the RuvC-like domain comprises a D10A, DBA, E762A, and/or D986A mutation and the HNH-like domain comprises a H840A, H559A, N854A, N865A, and/or N863A mutation. Alternatively, the catalytically inactive CRISPR/Cas protein can be a catalytically inactive (dead) Cpf1 protein comprising comparable mutations in the nuclease domain.

The at least one non-nuclease domain linked to the catalytically inactive CRISPR/Cas protein can be a transcription activation domain, a transcription repressor domain, or an epigenetic modification domain.

In some embodiments, the catalytically inactive CRISPR/Cas protein can be linked to at least one transcription activation domain. Suitable transcription activation domains include, without limit, herpes simplex virus VP16 domain, VP64 (which is a tetrameric derivative of VP16), VP160 10×VP16), p65 activation domain from NFκB, heat-shock factor 1 (HSF1) activation domain, MyoD1 activation domain, GCN4 peptide, 10×GCN4, viral R transactivator (Rta), VPR (a fusion of VP64-p65-Rta), p53 activation domains 1 and 2, CREB (cAMP response element binding protein) activation domains, E2A activation domains, or nuclear factor of activated T-cells (NFAT) activation domains. In some instances, the catalytically inactive CRISPR/Cas protein can be linked to one transcription activation domain, two transcription activation domains, three transcription activation domains, or more than three transcription activation domains.

In other embodiments, the catalytically inactive CRISPR/Cas protein can be linked to at least one transcription repressor domain. Non-limiting examples of suitable transcription repressor domains include Kruppel-associated box (KRAB) repressor domains, inducible cAMP early repressor (ICER) domains, YY1 glycine rich repressor domains, Sp1-like repressors, E(spI) repressors, IκB repressor, or methyl-CpG binding protein 2 (MeCP2) repressor domain. In some instances, the catalytically inactive CRISPR/Cas protein can be linked to one transcription repressor domain, two transcription repressor domains, three transcription repressor domains, or more than three transcription repressor domains.

In further embodiments, the catalytically inactive CRISPR/Cas protein can be linked at least one epigenetic modification domain. Epigenetic modification domains can alter DNA or chromatin structure (and may or may not alter DNA sequence). Non-limiting examples of suitable epigenetic modification domains include those with DNA methyltransferase activity (e.g., cytosine methyltransferase), DNA demethylase activity, DNA deamination (e.g., cytosine deaminase, adenosine deaminase, guanine deaminase), DNA amination, DNA oxidation activity, DNA helicase activity, histone acetyltransferase (HAT) activity (e.g., HAT domain derived from E1A binding protein p300), histone deacetylase activity, histone methyltransferase activity, histone demethylase activity, histone kinase activity, histone phosphatase activity, histone ubiquitin ligase activity, histone deubiquitinating activity, histone adenylation activity, histone deadenylation activity, histone SUMOylating activity, histone deSUMOylating activity, histone ribosylation activity, histone deribosylation activity, histone myristoylation activity, histone demyristoylation activity, histone citrullination activity, histone alkylation activity, histone dealkylation activity, histone oxidation activity, or histone interacting/remodeling activity. In specific embodiments, the epigenetic modification domain can comprise cytidine deaminase activity, histone acetyltransferase activity, or DNA methyltransferase activity. In particular embodiments, the epigenetic modification domain can be p300 histone acetyltransferase, activation-induced cytidine deaminase (AID), APOBEC cytidine deaminase, or TET methylcytosine dioxygenase. In some instances, the catalytically inactive CRISPR/Cas protein can be linked to one epigenetic modification domain, two epigenetic modification domains, three epigenetic modification domains, or more than three epigenetic modification domains.

The catalytically inactive CRISPR/Cas protein can be linked to the least one non-nuclease domain directly via chemical bonds or indirectly via linkers. The chemical bond can be covalent (e.g., peptide bond, ester bond, and the like). Alternatively, the chemical bond can be non-covalent (e.g., ionic, electrostatic, hydrogen, hydrophobic, Van der Waals interactions, or 7-effects). Suitable linkers are described above in section (II)(a)(iii). The at least one non-nuclease domain can be linked to the N-terminus, the C-terminus, and/or an internal location of the catalytically inactive CRISPR/Cas protein.

Optional Protein Domains. The catalytically inactive CRISPR/Cas protein linked to the at least non-nuclease domain can further comprise to at least one at least one nuclear localization signal (NLS), cell penetrating peptide (CPP), and/or marker domain. Examples of suitable NLSs, CPPs, and marker domains as detailed above in section (II)(b)(i). The at least one NLS, CPP, and/or marker domain can be linked directly or indirectly to the N-terminus, the C-terminus, and/or an internal location of the CRISPR/Cas protein having non-nuclease activity.

In some embodiments, CRISPR/Cas protein having non-nuclease activity can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a hapten (e.g., biotin, digoxigenin, and the like), quantum dots, or gold particles.

### (III) Kits

Still another aspect of the present disclosure provides kits comprising the aptamer-tracrRNAs, the synthetic two-part guide RNAs, the RNA aptamer binding proteins, and/or the CRISPR/Cas proteins disclosed herein.

In some embodiments, the kits can comprise at least one of the aptamer-tracrRNAs, as described above in section (I)(b), or nucleic acid encoding the at least one aptamer-tracrRNA, as described below in section (IV). In other embodiments, the kits can comprise at least one aptamer-tracrRNA (or encoding nucleic acid) and at least one RNA aptamer binding protein, as described above in section (II)(a), or nucleic acid encoding the at least one RNA aptamer binding protein, as described below in section (IV). In yet other embodiments, the kits can comprise at least one aptamer-tracrRNA (or encoding nucleic acid), at least one RNA aptamer binding protein (or encoding nucleic acid), and at least one CRISPR/Cas protein, as described above in section (II)(b), or nucleic acid encoding the at least one CRISPR/Cas protein. Any of these kits can further comprise at least one crRNA (e.g., a library of crRNAs) or nucleic acid encoding said crRNA. Alternatively, the end user can provide the at least one crRNA to be used in conjunction with the aptamer-tracrRNA(s) in the kit.

In other embodiments, the kits can comprise at least one of the synthetic two-part guide RNAs, as described above in section (I). In other embodiments, the kits can comprise at least one synthetic two-part guide RNA and at least one RNA aptamer binding protein, as described above in section (II)(a), or nucleic acid encoding the at least one RNA aptamer binding protein, as described below in section (IV). In yet other embodiments, the kits can comprise at least one synthetic two-part guide RNAs, at least one RNA aptamer binding protein (or encoding nucleic acid), and at least one CRISPR/Cas protein, as described above in section (II)(b), or nucleic acid encoding the at least one CRISPR/Cas protein.

The kits can further comprise transfection reagents, cell growth media, selection media, in-vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### (IV) Nucleic Acids

A further aspect of the present disclosure provides nucleic acids encoding the aptamer-tracrRNAs, the synthetic two-part guide RNAs, the RNA aptamer binding proteins, and/or the CRISPR/Cas proteins disclosed herein. The nucleic acids can be DNA or RNA, linear or circular, single-stranded or double-stranded. The nucleic acids encoding the CRISPR/Cas proteins can be codon optimized for efficient translation into protein in the eukaryotic cell of interest. Codon optimization programs are available as freeware or from commercial sources.

In some embodiments, the nucleic acid(s) encoding the aptamer-tracrRNA(s) can be DNA. The DNA encoding the aptamer-tracrRNA can be operably linked to a promoter sequence that is recognized by a phage RNA polymerase for in vitro RNA synthesis. For example, the promoter sequence can be a T7, T3, or SP6 promoter sequence or a variation of a T7, T3, or SP6 promoter sequence. In other embodiments, the DNA encoding the aptamer-tracrRNA can be operably linked to a promoter sequence for expression in eukaryotic cells. For example, DNA encoding the aptamer-tracrRNA(s) can be operably linked to a promoter sequence that is recognized by RNA polymerase III (Pol III). Examples of suitable Pol III promoters include, but are not limited to, mammalian U6, U3, H1, and 7SL RNA promoters. The DNA encoding the aptamer-tracrRNA can be part of a vector, as detailed below. Similarly, DNA encoding the crRNA(s) can be operably linked to phage promoter sequences and/or Pol III promoter sequences.

In further embodiments, the nucleic acid(s) encoding the at least one RNA aptamer binding protein and/or the CRISPR/Cas protein(s) can be RNA. The RNA can be enzymatically synthesized in vitro. For this, DNA encoding the RNA aptamer binding protein(s) or the CRISPR/Cas protein can be can be operably linked to a phage promoter sequence, as described above. In such embodiments, the in vitro-transcribed RNA can be purified, capped, and/or polyadenylated.

In other embodiments, the RNA encoding the RNA aptamer binding protein and/or the CRISPR/Cas protein can be part of a self-replicating RNA (Yoshioka et al., Cell Stem Cell, 2013, 13:246-254). The self-replicating RNA can be derived from a noninfectious, self-replicating Venezuelan equine encephalitis (VEE) virus RNA replicon, which is a positive-sense, single-stranded RNA that is capable of self-replicating for a limited number of cell divisions, and which can be modified to code proteins of interest (Yoshioka et al., Cell Stem Cell, 2013, 13:246-254).

In still other embodiments, the nucleic acid(s) encoding the RNA aptamer binding protein and/or the CRISPR/Cas protein(s) can be DNA. The DNA coding sequence encoding can be operably linked to at least one promoter control sequence for expression in the cell of interest. In certain embodiments, the DNA coding sequence can be operably linked to a promoter sequence for expression of the RNA aptamer binding protein or the CRISPR/Cas protein in bacterial (e.g., E. coli) cells or eukaryotic (e.g., yeast, insect, or mammalian) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. The promoter sequence can be wild type or it can be modified for more efficient or efficacious expression. In some embodiments, the DNA coding sequence also can be linked to a polyadenylation signal (e.g., SV40 polyA signal, bovine growth hormone (BGH) polyA signal, etc.) and/or at least one transcriptional termination sequence. In some situations, the RNA aptamer binding protein(s) and/or the CRISPR/Cas protein can be purified from the bacterial or eukaryotic cells.

In various embodiments, nucleic acid encoding the aptamer-tracrRNAs, RNA aptamer binding proteins, and/or CRISPR/Cas proteins can be present in a vector. Suitable vectors include plasmid vectors, viral vectors, and self-replicating RNA (Yoshioka et al., Cell Stem Cell, 2013, 13:246-254). In some embodiments, the encoding nucleic acid can be present in a plasmid vector. Non-limiting examples of suitable plasmid vectors include pUC, pBR322, pET, pBluescript, and variants thereof. In other embodiments, the encoding nucleic acid can be part of a viral vector (e.g., lentiviral vectors, adeno-associated viral vectors, adenoviral vectors, and so forth). The plasmid or viral vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

### (V) Methods for Targeted Transcriptional Regulation, Targeted Epigenome Modification, or Targeted Genome Modification

Another aspect of the present disclosure encompasses methods for targeted transcription activation, targeted transcription repression, targeted epigenome modification, or targeted genome modification, wherein the method comprises introducing into the cell any of the synthetic two-part guide RNA described above in section (I), at least one RNA aptamer binding protein as defined above in section (II)(a) or nucleic acid encoding the at least one RNA aptamer binding protein, and a CRISPR/Cas protein as defined above in section (II)(b) or nucleic acid encoding the CRISPR/Cas protein. In the methods disclosed herein, the efficiency and/or specificity of targeted transcription activation, targeted transcription repression, targeted epigenome modification, or targeted genome modification is increased relative to a CRISPR/Cas system in which the tracrRNA does not contain an RNA aptamer sequence. Additionally, in embodiments in which the aptamer-tracrRNA further comprises an extension sequence, the efficiency of targeted transcription activation, targeted transcription repression, targeted epigenome modification, or targeted genome modification is increased relative to an aptamer-tracrRNA that does not contain an extension sequence.

The gRNA guides the CRISPR/Cas protein to the target sequence in the chromosomal DNA. To accomplish this, the crRNA hybridizes with both the target chromosomal sequence and the tracrRNA, which also interacts with the CRISPR/Cas protein. Moreover, the at least one RNA aptamer binding protein binds/interacts with the at least one least one RNA aptamer sequence in the tracrRNA, thereby allowing the effector domains associated with the RNA aptamer binding protein to interact with the chromosomal DNA, proteins associated with the chromosomal DNA, and/or the CRISPR/Cas protein. As a consequence of these interactions, the effectiveness and/or specificity of the CRISPR/Cas protein-mediated targeted transcription activation, targeted transcription repression, targeted epigenome modification, or targeted genome modification genome is increased.

In some embodiments, the method can be modified for multiplexed applications, wherein the method further comprises introducing additional crRNAs into the eukaryotic cell. Each crRNA has a different 5′ sequence (i.e., is targeted to a different chromosomal sequence), but has a universal 3′ sequence such that it can base pair with the tracrRNA.

In embodiments in which the CRISPR/Cas protein is a catalytically inactive CRISPR/Cas protein linked to at least one transcription activation domain, transcription repressor domain, or epigenome modification domain, transcription of the target chromosomal sequence can be modified, histones/nucleosomes can be modified (e.g., acetylation, methylation, phosphorylation, adenylation, and the like), or DNA can be modified (e.g., methylation, deamination, and so forth). The frequency and/or efficiency of such modifications are increased relative to a CRISPR/Cas system in which the tracrRNA does not contain an RNA aptamer sequence (or an aptamer-tracrRNA that does not contain an extension sequence) (see the Examples).

In embodiments in which the CRISPR/Cas protein comprises nuclease activity, the CRISPR/Cas nuclease can cleave both strands of the double-stranded chromosomal sequence (i.e., generates a double-stranded break). The double-stranded break in the chromosomal sequence can be repaired by a non-homologous end-joining (NHEJ) repair process. Because NHEJ is error-prone, indels (i.e., deletions or insertions) of at least one base pair, substitutions of at least one base pair, or combinations thereof can occur during the repair of the break. Accordingly, the targeted chromosomal sequence can be modified, mutated, or inactivated. For example, a deletion, insertion, or substitution in the reading frame of a coding sequence can lead to an altered protein product, or no protein product (which is termed a “knock out”). In some iterations, the method can further comprise introducing into the cell a donor polynucleotide (see below) comprising at least one donor sequence that is flanked by sequence having substantial sequence identity to sequences located on either side of the target chromosomal sequence, such that during repair of the double-stranded break by a homology directed repair process (HDR) the donor sequence in the donor polynucleotide can be exchanged with or integrated into the chromosomal sequence at the target chromosomal sequence. Integration of an exogenous sequence is termed a “knock in.” The frequency and/or efficiency of such targeted genome modifications are increased relative to a CRISPR/Cas system in which the tracrRNA does not contain an RNA aptamer sequence (or an aptamer-tracrRNA that does not contain an extension sequence).

(a) Introduction into the Cell

As mentioned above, the method comprises introducing into the cell at least one synthetic two-part gRNA, at least one RNA aptamer binding protein or encoding nucleic acid, and a CRISPR/Cas protein or encoding nucleic acid. The various molecules can be introduced into the cell of interest by a variety of means.

In some embodiments, the cell can be transfected with the appropriate molecules (i.e., protein, DNA, and/or RNA). Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001). In other embodiments, the molecules can be introduced into the cell by microinjection. For example, the molecules can be injected into the cytoplasm or nuclei of the cells of interest. The amount of each molecule introduced into the cell can vary, but those skilled in the art are familiar with means for determining the appropriate amount.

The various molecules can be introduced into the cell simultaneously or sequentially. For example, the nucleic acid encoding the at least one RNA aptamer binding protein and the CRISPR/Cas protein can be stably introduced in to the cell. Alternatively, all the components can be introduced into at the same time.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

(b) Optional Donor Polynucleotide

In embodiments in which the CRISPR/Cas protein has nuclease activity, the method can further comprise introducing at least one donor polynucleotide into the cell. The donor polynucleotide can be single-stranded or double-stranded, linear or circular, and/or RNA or DNA. In some embodiments, the donor polynucleotide can be a vector, e.g., a plasmid vector.

The donor polynucleotide comprises at least one donor sequence. In some aspects, the donor sequence of the donor polynucleotide can be a modified version of an endogenous or native chromosomal sequence. For example, the donor sequence can be essentially identical to a portion of the chromosomal sequence at or near the sequence targeted by the DNA modification protein, but which comprises at least one nucleotide change. Thus, upon integration or exchange with the native sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the “gene correction” integration of the modified sequence, the cell can produce a modified gene product from the targeted chromosomal sequence.

In other aspects, the donor sequence of the donor polynucleotide can be an exogenous sequence. As used herein, an “exogenous” sequence refers to a sequence that is not native to the cell, or a sequence whose native location is in a different location in the genome of the cell. For example, the exogenous sequence can comprise protein coding sequence, which can be operably linked to an exogenous promoter control sequence such that, upon integration into the genome, the cell is able to express the protein coded by the integrated sequence. Alternatively, the exogenous sequence can be integrated into the chromosomal sequence such that its expression is regulated by an endogenous promoter control sequence. In other iterations, the exogenous sequence can be a transcriptional control sequence, another expression control sequence, an RNA coding sequence, and so forth. As noted above, integration of an exogenous sequence into a chromosomal sequence is termed a “knock in.”

As can be appreciated by those skilled in the art, the length of the donor sequence can and will vary. For example, the donor sequence can vary in length from several nucleotides to hundreds of nucleotides to hundreds of thousands of nucleotides.

Typically, the donor sequence in the donor polynucleotide is flanked by an upstream sequence and a downstream sequence, which have substantial sequence identity to sequences located upstream and downstream, respectively, of the sequence targeted by the CRISPR/Cas protein. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the sequence targeted by the CRISPR/Cas protein. Similarly, the downstream sequence refers to a nucleic acid sequence that shares substantial sequence identity with a chromosomal sequence downstream of the sequence targeted by the CRISPR/Cas protein. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the target sequence. In an exemplary embodiment, the upstream and downstream sequences in the donor polynucleotide can have about 95% or 100% sequence identity with chromosomal sequences upstream or downstream to the sequence targeted by the CRISPR/Cas protein.

In some embodiments, the upstream sequence shares substantial sequence identity with a chromosomal sequence located immediately upstream of the sequence targeted by the CRISPR/Cas protein. In other embodiments, the upstream sequence shares substantial sequence identity with a chromosomal sequence that is located within about one hundred (100) nucleotides upstream from the target sequence. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the target sequence. In some embodiments, the downstream sequence shares substantial sequence identity with a chromosomal sequence located immediately downstream of the sequence targeted by the CRISPR/Cas protein. In other embodiments, the downstream sequence shares substantial sequence identity with a chromosomal sequence that is located within about one hundred (100) nucleotides downstream from the target sequence. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the target sequence.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In specific embodiments, upstream and downstream sequences can range in length from about 50 to about 1500 nucleotides.

(c) Targeted Transcriptional Regulation, Epigenome Modification, or Genome Modification

Interactions between the crRNA and target chromosomal DNA, between the crRNA and the tracrRNA, between the crRNA/tracrRNA and CRISPR/Cas protein, between the RNA aptamer binding protein and RNA aptamer sequence(s) in the tracrRNA, and between the effector domain(s) linked to the RNA aptamer binding protein and the target chromosomal sequence, proteins associated with the target chromosomal sequence and/or the CRISPR/Cas protein facilitate and increase the efficiency of the targeted transcription regulation, targeted epigenome modification, or targeted genome modification.

In various iterations, the efficiency of targeted transcription activation, targeted transcription repression, targeted epigenome modification, or targeted genome modification genome can be increased by at least about 0.1-fold, at least about 0.5-fold, at least about 1-fold, at least about 2-fold, at least about 5-fold, at least about 10-fold, or at least about 20-fold, at least about 50-fold, at least about 100-fold, or more than about 100-fold relative to a CRISPR/Cas system in which the tracrRNA comprises no RNA aptamer sequences (or an aptamer-tracrRNA that does not contain an extension sequence).

(d) Cell Types

A variety of cells are suitable for use in the methods disclosed herein. In general, the cell is a eukaryotic cell. For example, the cell can be a human mammalian cell, a non-human mammalian cell, a non-mammalian vertebrate cell, an invertebrate cell, an insect cell, a plant cell, a yeast cell, or a single cell eukaryotic organism. In some embodiments, the cell can also be a one cell embryo. For example, a non-human mammalian embryo including rat, hamster, rodent, rabbit, feline, canine, ovine, porcine, bovine, equine, and primate embryos. In still other embodiments, the cell can be a stem cell such as embryonic stem cells, ES-like stem cells, fetal stem cells, adult stem cells, and the like. In one embodiment, the stem cell is not a human embryonic stem cell. Furthermore, the stem cells may include those made by the techniques disclosed in WO2003/046141, which is incorporated herein in its entirety, or Chung et al. (Cell Stem Cell, 2008, 2:113-117). The cell can be in vitro or in vivo (i.e., within an organism). In specific embodiments, the cell is a mammalian cell or mammalian cell line. In particular embodiments, the cell is a human cell or human cell line.

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Nepal c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells. An extensive list of mammalian cell lines may be found in the American Type Culture Collection catalog (ATCC, Manassas, Va.).

### (VI) Methods for Detecting Specific Genomic Loci

In embodiments in which the CRISPR/Cas protein has non-nuclease activity, the method detailed above can be modified for detecting or visualizing specific genomic loci in eukaryotic cells. In such embodiments, the CRISPR/Cas protein further comprises at least one detectable label. The detectable label can be a fluorophore (e.g., FAM, TMR, Cy3, Cy5, Texas Red, Oregon Green, Alexa Fluors, Halo tags, or suitable fluorescent dye), a purification tag (e.g., biotin, digoxigenin, and the like), quantum dots, or gold particles. The interactions between the various components disclosed herein facilitate and enhance detection of specific genomic loci or targeted chromosomal sequences.

The method comprises introducing into the eukaryotic cell at least one synthetic two-part gRNA, at least one RNA aptamer binding protein or encoding nucleic acid, and a detectably labeled CRISPR/Cas protein or encoding nucleic acid, and detecting the labeled CRISPR/Cas bound to the target chromosomal sequence. The detecting can be via dynamic live cell imaging, fluorescent microscopy, confocal microscopy, immunofluorescence, immunodetection, RNA-protein binding, protein-protein binding, and the like. The detecting step can be performed in live cells or fixed cells.

In embodiments in which the method comprises detecting chromatin structural dynamics in live cells, the components can be introduced into the cell as proteins or nucleic acids. In embodiments in which the method comprises detecting the targeted chromosomal sequence in fixed cells, the components can be introduced into the cell as proteins (or RNA-protein complexes). Means for fixing and permeabilizing cells are well known in the art. In some embodiments, the fixed cells can be subjected to chemical and/or thermal denaturation processes to convert double-stranded chromosomal DNA into single-stranded DNA. In other embodiments, the fixed cells are not subjected to chemical and/or thermal denaturation processes.

In embodiments, the guide RNA can further comprise a detectable label for in situ detection (e.g., FISH or CISH). Detectable labels are known in the art.

### (VII) Applications

The compositions and methods disclosed herein can be used in a variety of therapeutic, diagnostic, industrial, and research applications. In some embodiments, the present disclosure can be used to modulate transcription of any chromosomal sequence or modify/edit any chromosomal sequence of interest in a cell, animal, or plant in order to model and/or study the function of genes, study genetic or epigenetic conditions of interest, or study biochemical pathways involved in various diseases or disorders. For example, transgenic organisms can be created that model diseases or disorders, wherein the expression of one or more nucleic acid sequences associated with a disease or disorder is altered. The disease model can be used to study the effects of mutations on the organism, study the development and/or progression of the disease, study the effect of a pharmaceutically active compound on the disease, and/or assess the efficacy of a potential gene therapy strategy.

In other embodiments, the compositions and methods can be used to perform efficient and cost effective functional genomic screens, which can be used to study the function of genes involved in a particular biological process and how any alteration in gene expression can affect the biological process, or to perform saturating or deep scanning mutagenesis of genomic loci in conjunction with a cellular phenotype. Saturating or deep scanning mutagenesis can be used to determine critical minimal features and discrete vulnerabilities of functional elements required for gene expression, drug resistance, and reversal of disease, for example.

In further embodiments, the compositions and methods disclosed herein can be used for diagnostic tests to establish the presence of a disease or disorder and/or for use in determining treatment options. Examples of suitable diagnostic tests include detection of specific mutations in cancer cells (e.g., specific mutation in EGFR, HER2, and the like), detection of specific mutations associated with particular diseases (e.g., trinucleotide repeats, mutations in β-globin associated with sickle cell disease, specific SNPs, etc.), detection of hepatitis, detection of viruses (e.g., Zika), and so forth.

In additional embodiments, the compositions and methods disclosed herein can be used to correct genetic mutations associated with a particular disease or disorder such as, e.g., correct globin gene mutations associated with sickle cell disease or thalassemia, correct mutations in the adenosine deaminase gene associated with severe combined immune deficiency (SCID), reduce the expression of HTT, the disease-causing gene of Huntington's disease, or correct mutations in the rhodopsin gene for the treatment of retinitis pigmentosa. Such modifications may be made in cells ex vivo.

In still other embodiments, the compositions and methods disclosed herein can be used to generate crop plants with improved traits or increased resistance to environmental stresses. The present disclosure can also be used to generate farm animal with improved traits or production animals. For example, pigs have many features that make them attractive as biomedical models, especially in regenerative medicine or xenotransplantation.

### Enumerated Embodiments

The following enumerated embodiments are presented to illustrate certain aspects of the present invention, and are not intended to limit its scope.

1. A synthetic two-part guide RNA (gRNA) comprising (a) a clustered regularly interspersed short palindromic repeats (CRISPR) RNA (crRNA) and (b) a transacting crRNA (tracrRNA), wherein the crRNA comprises a 5′ sequence that is complementary to a target sequence in chromosomal DNA and a 3′ sequence that is capable of base pairing with a portion of the tracrRNA; and the tracrRNA comprises a 5′ tetraloop and at least one stem-loop, wherein the 5′ tetraloop and/or at least one stem-loop is modified to contain at least one hairpin-forming RNA aptamer sequence.

2. The synthetic two-part gRNA of enumeration 1, wherein the at least one hairpin-forming RNA aptamer sequence is MS2 sequence, PP7 sequence, com sequence, box B sequence, histone mRNA 3′ sequence, AU-rich element (ARE) sequence, or variants thereof.

3. The synthetic two-part gRNA of enumeration 1 or 2, wherein the at least one hairpin-forming RNA aptamer sequence is located in the 5′ tetraloop, in the at least one stem-loop, and/or at the 3′ end of the tracrRNA.

4. The synthetic two-part gRNA of enumeration 3, wherein the tracrRNA comprises stem-loop 1, stem-loop 2, and stem-loop 3, and at least one hairpin-forming RNA aptamer is located in the 5′ tetraloop and/or stem-loop 2.

5. The synthetic two-part gRNA of enumeration 4, wherein the 5′ tetraloop and/or stem-loop 2 further comprises an extension sequence.

6. The synthetic two-part gRNA of enumeration 5, wherein the extension sequence comprises from about 2 nucleotides to about 30 nucleotides.

7. The synthetic two-part gRNA of enumerations 5 or 6, wherein the crRNA further comprises a sequence that is capable of base paring with the extension sequence in the 5′ tetraloop or a portion of the extension sequence in the 5′ tetraloop of the tracrRNA.

8. The synthetic two-part gRNA of any one of enumerations 1 to 7, wherein the crRNA is chemically synthesized

9. The synthetic two-part gRNA of any one of enumerations 1 to 7, wherein the tracrRNA is enzymatically synthesized in vitro.

10. A nucleic acid encoding the tracrRNA of any one of enumerations 1 to 6.

11. The nucleic acid of enumeration 9, which is operably linked to a promoter sequence that is recognized by a phage RNA polymerase for in vitro RNA synthesis.

12. The nucleic acid of enumerations 9 or 10, which is part of a vector.

13. A kit comprising a tracrRNA as defined in any one of enumerations 1 to 6 or a nucleic acid as defined in any one of enumerations 10 to 12.

14. The kit of enumeration 13, further comprising at least one crRNA as defined in any one of enumerations 1, 7, or 8.

15. The kit of enumerations 13 or 14, wherein the at least one crRNA comprises a library of crRNA.

16. The kit of any one of enumerations 13 to 15, further comprising at least RNA aptamer binding protein associated with at least one functional domain or nucleic acid encoding the at least one RNA aptamer binding protein associated with at least one functional domain.

17. The kit of enumeration 16, wherein the RNA aptamer binding protein is MCP, PCP, Com, N22, SLBP, or FXR1, and the at least one functional domain is a transcription activation domain, a transcription repressor domain, an epigenetic modification domain, a marker domain, or combination thereof.

18. The kit of enumeration 17, wherein the transcription activation domain is VP16 activation domain, VP64 activation domain, VP160 activation domain, p65 activation domain from NFκB, heat-shock factor 1 (HSF1) activation domain, MyoD1 activation domain, GCN4 peptide, viral R transactivator (Rta), 53 activation domain, cAMP response element binding protein (CREB) activation domain, E2A activation domain, or nuclear factor of activated T-cells (NFAT) activation domain.

19. The kit of enumeration 17, wherein the transcription repressor domain is Kruppel-associated box (KRAB) repressor domain, inducible cAMP early repressor (ICER) domain, YY1 glycine rich repressor domain, Sp1-like repressor domain, E(spI) repressor domain, IκB repressor domain, or methyl-CpG binding protein 2 (MeCP2) repressor domain.

20. The kit of enumeration 17, wherein the epigenetic modification domain has acetyltransferase activity, deacetylase activity, methyltransferase activity, demethylase activity, kinase activity, phosphatase activity, amination activity, deamination activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, citrullination activity, alkylation activity, dealkylation activity, helicase activity, oxidation activity, or nucleosome interacting activity.

21. The kit of enumeration 20, wherein the epigenetic modification domain is p300 histone acetyltransferase, activation-induced cytidine deaminase (AID), APOBEC cytidine deaminase, or TET methylcytosine dioxygenase.

22. The kit of enumeration 17, wherein the marker domain is a fluorescent protein, a purification tag, or an epitope tag.

23. The kit of any one of enumeration 13 to 22, further comprising a CRISPR/Cas protein or nucleic acid encoding the CRISPR/Cas protein.

24. The kit of enumeration 23, wherein the CRISPR/Cas protein has nuclease activity or the CRISPR/Cas protein has non-nuclease activity.

25. The kit of enumeration 24, wherein the CRISPR/Cas protein having nuclease activity is a CRISPR/Cas nuclease or a catalytically inactive CRISPR/Cas protein linked to a non-CRISPR/Cas nuclease domain.

26. The kit of enumeration 24, wherein the CRISPR/Cas protein having non-nuclease activity is a catalytically inactive CRISPR/Cas protein linked to a non-nuclease domain.

27. The kit of enumeration 26, wherein the non-nuclease domain is a transcription activation domain, a transcription repressor domain, or an epigenetic modification domain.

28. The kit of enumeration 27, wherein the transcription activation domain is VP16 activation domain, VP64 activation domain, VP160 activation domain, NFκB p65 activation domain, heat-shock factor 1 (HSF1) activation domain, MyoD1 activation domain, GCN4 peptide, viral R transactivator (Rta), 53 activation domain, cAMP response element binding protein (CREB) activation domain, E2A activation domain, or nuclear factor of activated T-cells (NFAT) activation domain.

29. The kit of enumeration 27, wherein the transcription repressor domain is Kruppel-associated box (KRAB) repressor domain, YY1 glycine rich repressor domain, Sp1-like repressor domain, E(spI) repressor domain, IκB repressor domain, or methyl-CpG binding protein 2 (MeCP2) repressor domain.

30. The kit of enumeration 27, wherein the epigenetic modification domain has acetyltransferase activity, deacetylase activity, methyltransferase activity, demethylase activity, kinase activity, phosphatase activity, amination activity, deamination activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, citrullination activity, alkylation activity, dealkylation activity, helicase activity, oxidation activity, or nucleosome interacting activity.

31. The kit of enumeration 30, wherein the epigenetic modification domain is p300 histone acetyltransferase, activation-induced cytidine deaminase (AID), APOBEC cytidine deaminase, or TET methylcytosine dioxygenase.

32. The kit of any one of enumerations 23 to 31, wherein the CRISPR/Cas protein is a type II CRISPR/Cas nuclease or a type V CRISPR/Cas nuclease.

33. The kit of any one of enumerations 23 to 32, wherein the CRISPR/Cas protein further comprises at least one nuclear localization signal, at least one cell penetrating peptide, at least one marker domain, or combination thereof.

34. A composition comprising (a) a synthetic two-part gRNA as defined in any one of enumerations 1 to 9; (b) at least one RNA aptamer binding protein as defined in any one of enumeration 16 to 22; and (c) a CRISPR/Cas protein as defined in any one of enumeration 23 to 33.

35. A method for targeted transcription activation, targeted transcription repression, targeted epigenome modification, targeted genome modification, or targeted genomic locus visualization in a eukaryotic cell, the method comprising introducing into the cell (a) a synthetic two-part gRNA as defined in any one of enumerations 1 to 9; (b) at least one RNA aptamer binding protein as defined in any one of enumerations 16 to 22; and (c) at least one CRISPR/Cas protein as defined in any one of enumerations 23 to 33.

36. The method of enumeration 35, wherein the combination of (a), (b), and (c) has increased efficiency and/or specificity relative to a CRISPR/Cas system in which the gRNA does not contain an RNA aptamer sequence.

37. The method of enumerations 35 or 36, wherein the method further comprises introducing one or more additional crRNAs, each additional crRNA comprising a different 5′ sequence but a universal 3′ sequence.

38. The method of any one of enumerations 35 to 37, wherein the CRISPR/Cas protein has nuclease activity and the method further comprises introducing into the eukaryotic cell a donor polynucleotide comprising at least one donor sequence.

39. The method of any one of enumerations 35 to 38, wherein the eukaryotic cell is in vitro.

40. The method of any one of enumerations 35 to 38, wherein the eukaryotic cell is in vivo.

41. The method of any one of enumerations 35 to 40, wherein the eukaryotic cell is a mammalian cell.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

As used herein, the term “CRISPR/Cas system” refers to a complex comprising a CRISPR/Cas protein (i.e., nuclease, nickase, or catalytically dead protein) and a guide RNA.

The term “endogenous sequence,” as used herein, refers to a chromosomal sequence that is native to the cell.

As used herein, the term “exogenous” refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The term “nickase” refers to an enzyme that cleaves one strand of a double-stranded nucleic acid sequence (i.e., nicks a double-stranded sequence). For example, a nuclease with double strand cleavage activity can be modified by mutation and/or deletion to function as a nickase and cleave only one strand of a double-stranded sequence.

The term “nuclease,” as used herein, refers to an enzyme that cleaves both strands of a double-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The terms “target sequence,” “target chromosomal sequence,” and “target site” are used interchangeably to refer to the specific sequence in chromosomal DNA to which the CRISPR/Cas protein is targeted, and the site at which the CRISPR/Cas protein mediates its activity.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1

### Design and Synthesis of Two-Part Guide RNAs

The two-part gRNA disclosed herein contains one crRNA, which is target specific, and one aptamer-tracrRNA, which comprises universal sequence. The sequence and secondary structure of a typical two-part gRNA for SpCas9 (design #1) is shown in FIG. 1. MS2 stem-loops sequences (34 nt each) have been inserted in the tetraloop and stem-loop 2. An extension sequence (underlined) has been inserted in the tetraloop. The crRNA contains 20 nt individual spacer (target specific) sequence. Table 1 presents the sequences of this and several other two-part gRNA designs (the tetraloop extension sequences are underlined). The crRNAs were chemically synthesized, and the aptamer-tracrRNAs were enzymatically synthesized in vitro.

TABLE 1
Two-Part gRNA Designs

| | | | | SEQ |
|---|---|---|---|---|
| | Design | | | ID |
| Design | Element | | Sequence (5′-3′) | NO: |
| #1 | 10 nt | crRNA | NNNNNNNNNNNNNNNNNNNNGUU | 38 |
| | tetra- | | UUAGAGCUAUGCUGUUUUG | |
| | loop | Aptamer- | GGCCAACAUGAGGAUCACCCAUG | 39 |
| | exten- | tracrRNA | UCUGCAGGGCCCAAAACAGCAUA | |
| | sion | | GCAAGUUAAAAUAAGGCUAGUCC | |
| | | | GUUAUCAACUUGGCCAACAUGAG | |
| | | | GAUCACCCAUGUCUGCAGGGCCA | |
| | | | AGUGGCACCGAGUCGGUGCUU | |
| #2 | 8 nt | crRNA | NNNNNNNNNNNNNNNNNNNNGUU | 40 |
| | tetra- | | UUAGAGCUAUGCUGUUU | |
| | loop | Aptamer- | GGCCAACAUGAGGAUCACCCAUG | 41 |
| | exten- | tracrRNA | UCUGCAGGGCCAAACAGCAUAGC | |
| | sion | | AAGUUAAAAUAAGGCUAGUCCGU | |
| | | | UAUCAACUUGGCCAACAUGAGGA | |
| | | | UCACCCAUGUCUGCAGGGCCAAG | |
| | | | UGGCACCGAGUCGGUGCUU | |
| #3 | 6 nt | crRNA | NNNNNNNNNNNNNNNNNNNNGUU | 42 |
| | tetra- | | UUAGAGCUAUGCUGU | |
| | loop | Aptamer- | GGCCAACAUGAGGAUCACCCAUG | 43 |
| | exten- | tracrRNA | UCUGCAGGGCCACAGCAUAGCAA | |
| | sion | | GUUAAAAUAAGGCUAGUCCGUUA | |
| | | | UCAACUUGGCCAACAUGAGGAUC | |
| | | | ACCCAUGUCUGCAGGGCCAAGUG | |
| | | | GCACCGAGUCGGUGCUU | |
| #4 | No | crRNA | NNNNNNNNNNNNNNNNNNNNGUU | 44 |
| | tetra- | | UUAGAGCUA | |
| | loop | Aptamer- | GGCCAACAUGAGGAUCACCCAUG | 45 |
| | exten- | tracrRNA | UCUGCAGGGCCUAGCAAGUUAAA | |
| | sion | | AUAAGGCUAGUCCGUUAUCAACU | |
| | | | UGGCCAACAUGAGGAUCACCCAU | |
| | | | GUCUGCAGGGCCAAGUGGCACCG | |
| | | | AGUCGGUGCUU | |
| #5 | 16 nt | crRNA | NNNNNNNNNNNNNNNNNNNNGUU | 46 |
| | tetra- | | UUAGAGCUAUGCUGUUUUG | |
| | loop | Aptamer- | AUCUGCUAGGCCAACAUGAGGAU | 47 |
| | exten- | tracrRNA | CACCCAUGUCUGCAGGGCCUAGC | |
| | sion | | AACAAAACAGCAUAGCAAGUUAAA | |
| | | | AUAAGGCUAGUCCGUUAUCAACU | |
| | | | UGGCCAACAUGAGGAUCACCCAU | |
| | | | GUCUGCAGGGCCAAGUGGCACCG | |
| | | | AGUCGGUGCUU | |

### Example 2

### Targeted Gene Activation with Two-Part Guide RNAs gRNA

Several of the two-part gRNAs described above in Example 1 were tested for activation of human POU5F1 and IL1B genes. Those tested were design #1 (contains extended tetraloop sequence), design #5 (contains longer extended tetraloop sequence, and design #4 (contains no extended tetraloop sequence). The control crRNA+tracrRNA (Syg) contains no aptamer sequence. Both POU5F1 and IL1B genes are known to be down-regulated or not expressed in HEK293 (human embryonic kidney) cells. A stable HEK293 cell line was created using Lentivirus-mediated insertion of VP64-dCas9 and MS2-HSF1-P65. Cells were transfected with 100 pmol two-part gRNA per 150,000 cells in a 12-well tissue culture plate using 3 μl of a transfection reagent. Target sequences for POU5F1 (GGAAAACCGGGAGACACAAC; SEQ ID NO:48) or IL1B (AAAGGGGAAAAGAGTATTGG; SEQ ID NO:49) were included in the synthetic crRNA, and complexed at a 1:1 molar ratio with the synthesized tracrRNA in 10 mM TRIS, pH 7.5 and 0.1 mM EDTA at 95° C. 2 min and cooled 0.5° C./sec to 12° C.

Gene expression was assayed by harvesting total RNA and performing multiplexed quantitative RT-PCR (qRT-PCR) using Taqman probes (Hs003005111_m1, Hs00174097_m1). Expression was normalized to the expression of the housekeeping gene PPIA. Negative controls were achieved by transfecting cells with pMAX-GFP DNA. Activation of targets was compared against the control crRNA+tracrRNA that does not contain the aptamer modifications.

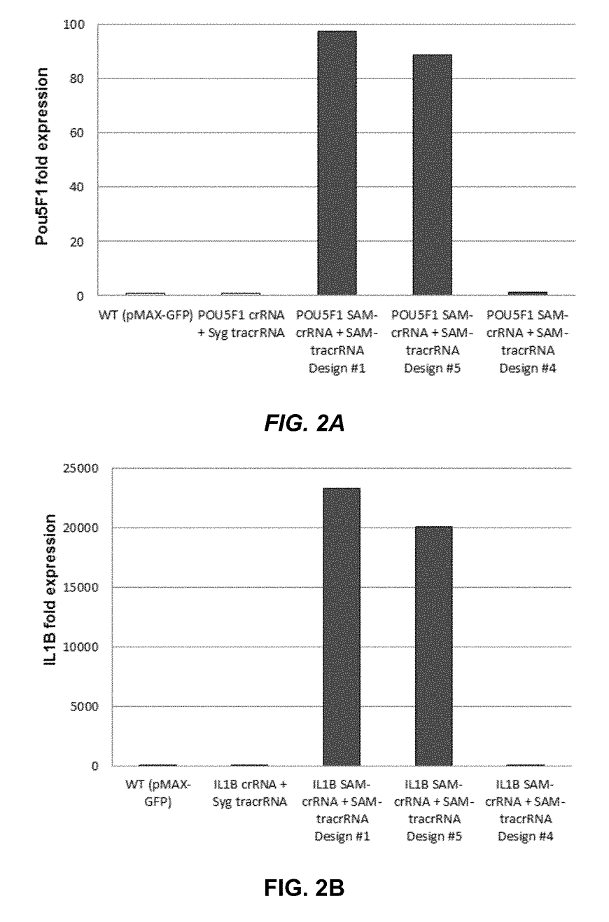

As shown in FIG. 2A and 2B, the two-part guide RNA design #1 and design #5 (both contain aptamer and extended tetraloop sequences) were able to promote transcriptional activation significantly on both POU5F1 (FIG. 2A) and IL1B (FIG. 2B), relative to negative controls. Significantly, the two-part guide RNA design #4 (contains aptamer sequence, but no extended tetraloop sequence) caused poor or no target activations. These data indicate that an extended tetraloop, as in design #1 or #5, is critical.

What is claimed is:

1. A synthetic two-part guide RNA (gRNA) comprising:
(a) a clustered regularly interspersed short palindromic repeats (CRISPR) RNA (crRNA); and
(b) a transacting crRNA (tracrRNA),
wherein:
the crRNA comprises a 5′ sequence that is complementary to a target sequence in chromosomal DNA and a 3′ sequence that is capable of base pairing with a portion of the tracrRNA; and
the tracrRNA comprises a 5′ tetraloop and at least one stem-loop, and the 5′ tetraloop and/or at least one stem-loop is modified to contain at least one hairpin-forming RNA aptamer sequence.

2. The synthetic two-part gRNA of claim 1, wherein the at least one hairpin-forming RNA aptamer sequence is MS2 sequence, PP7 sequence, com sequence, box B sequence, histone mRNA 3′ sequence, AU-rich element (ARE) sequence, or variants thereof.

3. The synthetic two-part gRNA of claim 1, wherein the at least one hairpin-forming RNA aptamer sequence is located in the 5′ tetraloop, in the at least one stem-loop, and/or at the 3′ end of the tracrRNA.

4. The synthetic two-part gRNA of claim 1, wherein the at least one stem-loop of the tracrRNA comprises stem-loop 1, stem-loop 2, and stem-loop 3, and the at least one hairpin-forming RNA aptamer sequence is located in the 5′ tetraloop and/or in stem-loop 2.

5. The synthetic two-part gRNA of claim 4, wherein the 5′ tetraloop and/or stem-loop 2 further comprises an extension sequence.

6. The synthetic two-part gRNA of claim 5, wherein the extension sequence comprises from about 2 nucleotides to about 30 nucleotides.

7. The synthetic two-part gRNA of claim 5, wherein the crRNA further comprises a sequence that is capable of base paring with the extension sequence in the 5′ tetraloop or a portion of the extension sequence in the 5′ tetraloop of the tracrRNA.

8. The synthetic two-part gRNA of claim 1, wherein the crRNA is chemically synthesized

9. The synthetic two-part gRNA of claim 1, wherein the tracrRNA is enzymatically synthesized in vitro.

10. A nucleic acid encoding the tracrRNA of claim 1.

11. The nucleic acid of claim 10, which is operably linked to a promoter sequence that is recognized by a phage RNA polymerase for in vitro RNA synthesis.

12. The nucleic acid of claim 10, which is part of a vector.

13. A method for targeted transcription activation, targeted transcription repression, targeted epigenome modification, targeted genome modification, or targeted genomic locus visualization in a eukaryotic cell, the method comprising introducing into the eukaryotic cell:
(a) a synthetic two-part gRNA as defined in claim 1;
(b) at least one RNA aptamer binding protein associated with at least one functional domain or nucleic acid encoding the at least one RNA aptamer binding protein associated with at least one functional domain; and
(c) at least one CRISPR/Cas protein or nucleic acid encoding the at least one CRISPR/Cas protein;
wherein interactions between (a), (b), (c), and the target sequence in chromosomal DNA leads to targeted transcription activation, targeted transcription repression, targeted epigenome modification, targeted genome modification, or targeted genomic locus visualization in the eukaryotic cell.

14. The method of claim 13, wherein the combination of (a), (b), and (c) has increased efficiency and/or specificity relative to a CRISPR/Cas system in which the gRNA does not contain an RNA aptamer sequence.

15. The method of claim 13, wherein the method further comprises introducing one or more additional crRNAs, each additional crRNA comprising a different 5′ sequence but a universal 3′ sequence.

16. The method of claim 13, wherein the at least one hairpin-forming RNA aptamer sequence of the tracrRNA is MS2 sequence, PP7 sequence, com sequence, box B sequence, histone mRNA 3′ sequence, AU-rich element (ARE) sequence, or variants thereof.

17. The method of claim 13, wherein the at least one hairpin-forming RNA aptamer sequence is located in the 5′ tetraloop, in the at least one stem-loop, and/or at the 3′ end of the tracrRNA.

18. The method of claim 13, wherein the at least one stem-loop of the tracrRNA comprises stem-loop 1, stem-loop 2, and stem-loop 3, and the at least one hairpin-forming RNA aptamer sequence is located in the 5′ tetraloop and/or in stem-loop 2.

19. The method of claim 13, wherein the 5′ tetraloop of the tracrRNA further comprises an extension sequence, and the crRNA further comprises a sequence that is capable of base paring with the extension sequence in the 5′ tetraloop or a portion of the extension sequence in the 5′ tetraloop of the tracrRNA.

20. The method of claim 13, wherein the at least one RNA aptamer binding protein is MCP, PCP, Com, N22, SLBP, or FXR1, and the at least one functional domain associated with the at least one RNA aptamer binding protein is a transcription activation domain, a transcription repressor domain, an epigenetic modification domain, a marker domain, or combination thereof.

21. The method of claim 20, wherein the transcription activation domain is VP16 activation domain, VP64 activation domain, VP160 activation domain, p65 activation domain from NFκB, or heat-shock factor 1 (HSF1) activation domain; the transcription repressor domain is Kruppel-associated box (KRAB) repressor domain; the epigenetic modification domain is p300 histone acetyltransferase, activation-induced cytidine deaminase (AID), APOBEC cytidine deaminase, TET methylcytosine dioxygenase, or has nucleosome interacting activity; and the marker domain is a fluorescent protein, a purification, or an epitope tag.

22. The method of claim 13, wherein the at least one CRISPR/Cas protein is a CRISPR/Cas nuclease or a catalytically inactive CRISPR/Cas protein linked to a non-CRISPR/Cas nuclease domain.

23. The method of claim 22, which further comprises introducing into the eukaryotic cell a donor polynucleotide comprising at least one donor sequence.

24. The method of claim 13, wherein the CRISPR/Cas protein is a catalytically inactive CRISPR/Cas protein linked to a non-nuclease domain, and the non-nuclease domain is a transcription activation domain, a transcription repressor domain, or an epigenetic modification domain.

25. The method of claim 13, wherein the at least one CRISPR/Cas protein is a type II Cas9 protein.

26. The method of claim 13, wherein the eukaryotic cell is in vitro.

27. The method of claim 13, wherein the eukaryotic cell is in vivo.

28. The method of claim 13, wherein the eukaryotic cell is a mammalian cell.


SEQUENCE LISTING (49 sequences)

SEQ ID NO: 1 - protein, 6 aa, Artificial Sequence
  SYNTHESIZED
LEGGGS

SEQ ID NO: 2 - protein, 4 aa, Artificial Sequence
  SYNTHESIZED
TGSG

SEQ ID NO: 3 - protein, 8 aa, Artificial Sequence
  SYNTHESIZED
GGSGGGSG

SEQ ID NO: 4 - protein, 20 aa, Artificial Sequence
  SYNTHESIZED
  MISC_FEATURE (6)..(20): can be either absent or present
GGGGSGGGGSGGGGSGGGGS

SEQ ID NO: 5 - protein, 8 aa, Artificial Sequence
  SYNTHESIZED
  MISC_FEATURES (7)..(8): can be either absent or present
GGGGGGGG

SEQ ID NO: 6 - protein, 20 aa, Artificial Sequence
  SYNTHESIZED
  MISC_FEATURE (6)..(20): can be either absent or present
EAAAKEAAAKEAAAKEAAAK

SEQ ID NO: 7 - protein, 5 aa, Artificial Sequence
  SYNTHESIZED
PAPAP

SEQ ID NO: 8 - protein, 7 aa, Artificial Sequence
  SYNTHESIZED
PKKKRKV

SEQ ID NO: 9 - protein, 7 aa, Artificial Sequence
  SYNTHESIZED
PKKKRRV

SEQ ID NO: 10 - protein, 16 aa, Artificial Sequence
  SYNTHESIZED
KRPAATKKAGQAKKKK

SEQ ID NO: 11 - protein, 11 aa, Artificial Sequence
  SYNTHESIZED
YGRKKRRQRRR

SEQ ID NO: 12 - protein, 9 aa, Artificial Sequence
  SYNTHESIZED
RKKRRQRRR

SEQ ID NO: 13 - protein, 9 aa, Artificial Sequence
  SYNTHESIZED
PAAKRVKLD

SEQ ID NO: 14 - protein, 11 aa, Artificial Sequence
  SYNTHESIZED
RQRRNELKRSP

SEQ ID NO: 15 - protein, 8 aa, Artificial Sequence
  SYNTHESIZED
VSRKRPRP

SEQ ID NO: 16 - protein, 8 aa, Artificial Sequence
  SYNTHESIZED
PPKKARED

SEQ ID NO: 17 - protein, 8 aa, Artificial Sequence
  SYNTHESIZED
PQPKKKPL

SEQ ID NO: 18 - protein, 12 aa, Artificial Sequence
  SYNTHESIZED
SALIKKKKKMAP

SEQ ID NO: 19 - protein, 7 aa, Artificial Sequence
  SYNTHESIZED
PKQKKRK

SEQ ID NO: 20 - protein, 10 aa, Artificial Sequence
  SYNTHESIZED
RKLKKKIKKL

SEQ ID NO: 21 - protein, 10 aa, Artificial Sequence
  SYNTHESIZED
REKKKFLKRR

SEQ ID NO: 22 - protein, 20 aa, Artificial Sequence
  SYNTHESIZED
KRKGDEVDGVDEVAKKKSKK

SEQ ID NO: 23 - protein, 17 aa, Artificial Sequence
  SYNTHESIZED
RKCLQAGMNLEARKTKK

SEQ ID NO: 24 - protein, 38 aa, Artificial Sequence
  SYNTHESIZED
NQSSNFGPMKGGNFGGRSSGPYGGGGQYFAKPRNQGGY

SEQ ID NO: 25 - protein, 42 aa, Artificial Sequence
  SYNTHEISZED
RMRIZFKNKGKDTAELRRRRVEVSVELRKAKKDEQILKRRNV

SEQ ID NO: 26 - protein, 20 aa, Artificial Sequence
  SYNTHESIZED
GRKKRRQRRRPPQPKKKRKV

SEQ ID NO: 27 - protein, 19 aa, Artificial Sequence
  SYNTHESIZED
PLSSIFSRIGDPPKKKRKV

SEQ ID NO: 28 - protein, 24 aa, Artificial Sequence
  SYNTHESIZED
GALFLGWLGAAGSTMGAPKKKRKV

SEQ ID NO: 29 - protein, 27 aa, Artificial Sequence
  SYNTHESIZED
GALFLGFLGAAGSTMGAWSQPKKKRKV

SEQ ID NO: 30 - protein, 21 aa, Artificial Sequence
  SYNTHESIZED
KETWWETWWTEWSQPKKKRKV

SEQ ID NO: 31 - protein, 11 aa, Artificial Sequence
  SYNTHESIZED
YARAAARQARA

SEQ ID NO: 32 - protein, 11 aa, Artificial Sequence
  SYNTEHSIZED
THRLPRRRRRR

SEQ ID NO: 33 - protein, 11 aa, Artificial Sequence
  SYNTHESIZED
GGRRARRRRRR

SEQ ID NO: 34 - protein, 12 aa, Artificial Sequence
  SYNTHESIZED
RRQRRTSKLMKR

SEQ ID NO: 35 - protein, 27 aa, Artificial Sequence
  SYNTHESIZED
GWTLNSAGYLLGKINLKALAALAKKIL

SEQ ID NO: 36 - protein, 33 aa, Artificial Sequence
  SYNTHESIZED
KALAWEAKLAKALAKALAKHLAKALAKALKCEA

SEQ ID NO: 37 - protein, 16 aa, Artificial Sequence
  SYNTHESIZED
RQIKIWFQNRRMKWKK

SEQ ID NO: 38 - RNA, 42 nt, Artificial Sequence
  SYNTHESIZED
  misc_feature (1)..(20): n is a, c, g, or u
nnnnnnnnnnnnnnnnnnnnguuuuagagcuaugcuguuuug

SEQ ID NO: 39 - RNA, 136 nt, Artificial Sequence
  SYNTHESIZED
ggccaacaugaggaucacccaugucugcagggcccaaaacagcauagcaaguuaaaauaaggcuaguccguuaucaacuuggccaacaugaggaucacccaugucugcagggccaaguggcaccgagucggugcuu

SEQ ID NO: 40 - RNA, 40 nt, Artificial Sequence
  SYNTHESIZED
  misc_feature (1)..(20): n is a, c, g, or u
nnnnnnnnnnnnnnnnnnnnguuuuagagcuaugcuguuu

SEQ ID NO: 41 - RNA, 134 nt, Artificial Sequence
  SYNTHESIZED
ggccaacaugaggaucacccaugucugcagggccaaacagcauagcaaguuaaaauaaggcuaguccguuaucaacuuggccaacaugaggaucacccaugucugcagggccaaguggcaccgagucggugcuu

SEQ ID NO: 42 - RNA, 38 nt, Artificial Sequence
  SYNTHESIZED
  misc_feature (1)..(20): n is a, c, g, or u
nnnnnnnnnnnnnnnnnnnnguuuuagagcuaugcugu

SEQ ID NO: 43 - RNA, 132 nt, Artificial Sequence
  SYNTHESIZED
ggccaacaugaggaucacccaugucugcagggccacagcauagcaaguuaaaauaaggcuaguccguuaucaacuuggccaacaugaggaucacccaugucugcagggccaaguggcaccgagucggugcuu

SEQ ID NO: 44 - RNA, 32 nt, Artificial Sequence
  SYNTHESIZED
  misc_feature (1)..(20): n is a, c, g, or u
nnnnnnnnnnnnnnnnnnnnguuuuagagcua

SEQ ID NO: 45 - RNA, 126 nt, Artificial Sequence
  SYNTHESIZED
ggccaacaugaggaucacccaugucugcagggccuagcaaguuaaaauaaggcuaguccguuaucaacuuggccaacaugaggaucacccaugucugcagggccaaguggcaccgagucggugcuu

SEQ ID NO: 46 - RNA, 42 nt, Artificial Sequence; identical to SEQ ID NO: 38
  SYNTHESIZED
  misc_feature (1)..(20): n is a, c, g, or u

SEQ ID NO: 47 - RNA, 150 nt, Artificial Sequence
  SYNTHESIZED
aucugcuaggccaacaugaggaucacccaugucugcagggccuagcaacaaaacagcauagcaaguuaaaauaaggcuaguccguuaucaacuuggccaacaugaggaucacccaugucugcagggccaaguggcaccgagucggugcuu

SEQ ID NO: 48 - DNA, 20 bp, Homo sapiens
ggaaaaccgggagacacaac

SEQ ID NO: 49 - DNA, 20 bp, Homo sapiens
aaaggggaaaagagtattgg